(19) United States
(12) Patent Application Publication
(10) Pub. No.: US 2023/0330132 A1
(43) Pub. Date: Oct. 19, 2023

(54) VIRAL INHIBITORS

(71) Applicant: NANOMERICS LIMITED, St Albans, Hertfordshire (GB)

(72) Inventors: Ijeoma UCHEGBU, St Albans, Hertfordshire (GB); Andreas SCHATZLEIN, St Albans, Hertfordshire (GB); Krzysztof PYRC, Krakow (PL); Aleksandra MILEWSKA, Krakow (PL); Emilia BARRETO DURAN, Krakow (PL)

(21) Appl. No.: 18/023,195

(22) PCT Filed: Aug. 24, 2021
(86) PCT No.: PCT/GB2021/052199

§ 371 Date: Feb. 24, 2023

(30) Foreign Application Priority Data
Aug. 24, 2020 (GB) 2013221.3
Dec. 3, 2020 (GB) 2019097.1
Apr. 15, 2021 (GB) 2105404.4

(51) Int. Cl. A61K 31/722; A61K 9/14; A61K 9/00; A61P 31/14

(52) U.S. Cl. CPC A61K 31/722; A61K 9/14; A61K 9/0073; A61P 31/14

(57) ABSTRACT

The present invention relates to a derivatised amphiphilic carbohydrate compound with molecular weight less than 50 kDa which is based on a glycol chitosan for use in the prevention or treatment of a viral infection. The invention further relates to the use of the compound when formulated with other components in pharmaceutical compositions for the prevention or treatment of viral infections, and further to a method of reducing viral replication in human tissues.

### FIELD OF THE INVENTION

The present invention relates to amphiphilic carbohydrate compounds and their use as antivirals.

### BACKGROUND OF THE INVENTION

There are no drug treatments to cure a wide variety of viral infections, such as infections involving the Ebola virus, coronaviruses or the influenza viruses and deadly respiratory viruses continue to cause epidemics and pandemics (Li, Q. et al. Early Transmission Dynamics in Wuhan, China, of Novel Coronavirus-Infected Pneumonia. N Engl J Med 382, 1199-1207, doi:10.1056/NEJMoa2001316 (2020)). For example, the full clinical spectrum of COVID-19 ranges from a mild, self-limiting respiratory tract illness to severe progressive viral pneumonia, multiorgan failure and death and seasonal influenza results in about 389,000 deaths worldwide every year (Li, Q. et al. Early Transmission Dynamics in Wuhan, China, of Novel Coronavirus-Infected Pneumonia. N Engl J Med 382, 1199-1207, doi:10.1056/NEJMoa2001316 (2020) and Paget, J. et al. Global mortality associated with seasonal influenza epidemics: New burden estimates and predictors from the GLaMOR Project. J Glob Health 9, 020421-020421, doi:10.7189/jogh.09.020421 (2019)).

Viral binding to respiratory epithelial cell-surface receptors is a critical step in the infection of host cells and the COVID-19 infectious agent (SARS-CoV-2) gains entry to the cell via the viral spike protein S1 receptor binding domain (RBD) interacting with the angiotensin converting enzyme-2 (ACE-2) receptor (Tai, W. et al. Characterization of the receptor-binding domain (RBD) of 2019 novel coronavirus: implication for development of RBD protein as a viral attachment inhibitor and vaccine. Cellular & Molecular Immunology, doi:10.1038/s41423-020-0400-4 (2020) and Yan, R. et al. Structural basis for the recognition of the SARS-CoV-2 by full-length human ACE2. Science, doi:10.1126/science.abb2762 (2020) and Wrapp, D. et al. Cryo-EM structure of the 2019-nCoV spike in the prefusion conformation. Science 367, 1260-1263, doi:10.1126/science.abb2507 (2020)).

The ACE-2 receptor is highly expressed in nasal epithelial cells (Sungnak, W. et al. SARS-CoV-2 entry factors are highly expressed in nasal epithelial cells together with innate immune genes. Nat. Med., doi:10.1038/s41591-020-0868-6 (2020)), making a nasal spray that inhibits viral entry into nasal epithelial cells an attractive prophylactic for use in the control of epidemics and pandemics. Viral receptor binding and internalisation occurs via an initial binding of the spike protein S1 binding domain to the ACE-2 receptor followed by a conformational change which results in membrane fusion of the viral particle with the epithelial cells and ultimately viral entry.

Polymers such as sulphated glycopolymers have been shown to inhibit the viral binding of human papillomavirus to cell surface receptors (Soria-Martinez, L. et al. Prophylactic Antiviral Activity of Sulfated Glycomimetic Oligomers and Polymers. Journal of the American Chemical Society 142, 5252-5265, doi:10.1021/jacs.9b13484 (2020)). Sulphated chitosan compounds, such as N-carboxymethylchitosan-N,O-sulfate were found to inhibit the synthesis of virus-specific proteins and the replication of HIV-1 in cultured T-cells as well as the replication of the Rausher murine leukemia virus in cultured mouse fibroblasts (Chirkov, S. N. [The antiviral activity of chitosan (review)]. Prikl Biokhim Mikrobiol 38, 5-13 (2002)). Additionally 6-deoxy-6-bromo-N-phthaloyl chitosan (He, X. et al. The improved antiviral activities of amino-modified chitosan derivatives on Newcastle virus. Drug and Chemical Toxicology, 1-6, doi:10.1080/01480545.2019.1620264 (2019)), and chitosan itself (Zheng, M. et al. Intranasal Administration of Chitosan Against Influenza A (H₇N9) Virus Infection in a Mouse Model. Scientific Reports 6, 28729, doi:10.1038/srep28729 (2016)) have been reported to have antiviral activity via a variety of mechanisms.

It is well known that quaternary ammonium compounds (QACs) are viricidal due to mechanisms involving viral cell lysis and DNA binding (Gerba, C. P. Quaternary ammonium biocides: efficacy in application. Appl Environ Microbiol 81, 464-469, doi:10.1128/AEM.02633-14 (2015)). However WO2013/172725 discloses that N-(2-hydroxypropyl)-3-trimethylammonium chitosan chloride (HTCC), a chitosan QAC with a molecular weight of 50-190 kDa (based on viscosity), and a level of quaternary ammonium groups ranging from 57% to 77%, inhibits coronavirus infections (e.g. HCOV-NL63) in vitro by a mechanism that involves an inhibition of viral entry into the cell (Milewska, A. et al. HTCC: Broad Range Inhibitor of Coronavirus Entry. PLoS One 11, e0156552, doi:10.1371/journal.pone.0156552 (2016) and Sigma Aldrich. Chitosan-Low Molecular Weight (Product Number 448869). https://www.sigmaaldrich.com/catalog/product/aldrich/448869?lang=en&region=GB&g clid=EAIalQobChMI8J7GkdG76gIVEO7tCH₃YZgTwEAAYAiAAEgKVKPD_BwE (Accessed 7 Jul. 2020)) and Milewska, A. et al. Novel polymeric inhibitors of HCoV-NL63. Antiviral Res 97, 112-121, doi:10.1016/j.antiviral.2012.11.006 (2013) and Ciejka, J., Wolski, K., Nowakowska, M., Pyrc, K. & Szczubialka, K. Biopolymeric nano/microspheres for selective and reversible adsorption of coronaviruses. Mater Sci Eng C Mater Biol Appl 76, 735-742, doi:10.1016/j.msec.2017.03.047 (2017)). It appears that HTCC prevents viral binding to the cell surface ACE-2 receptor, by electrostatic binding of positively charged HTCC to the viruses. This results in a Log Reduction Value of −4 in mammalian cells. It has also been shown that HTCC inhibits the entry of SARS-COV-2 into cells (Milewska, A. et al. HTCC as a highly effective polymeric inhibitor of SARS-CoV-2 and MERS-CoV. bioRxiv https://doi.org/10.1101/2020.03.29.014183 (2020)). The HTCC disclosed has a relatively high molecular weight (50 - 190 kDa) and a high level of quaternary ammonium group substitution (57-77mole %).

The quaternary ammonium group on HTCC is not the only factor essential for activity, as the following compounds were earlier found in Milewska, A. et al. Novel polymeric inhibitors of HCoV-NL63. Antiviral Res 97, 112-121, doi:10.1016/j.antivira1.2012.11.006 (2013), to be non-active in inhibiting coronavirus entry into cells despite the presence of a quaternary ammonium group: O-(2-hydroxypropyl)-3-trimethylammonium poly(vinyl alcohol) chloride (HTPVA), N-(2-hydroxypropy)-3-trimethylammonium dextran chloride, hydroxypropylcellulose-graft-poly(N-acrylamidopropyl-N,N,N- trimethylammonium chloride) (HPC-APTMAC), N-(3-ethylammonium) poly(allylamine) chloride, poly(methacryloyl aminopropyltrimethylammonium chloride) labeled with pyrene, copolymers of thymylethyl acrylate (TEA) with methacryloyl aminopropyltrimethylammonium chloride (MAPTAC) 25:75 and copolymers of thymylethyl acrylate (TEA) with methacryloyl aminopropyltrimethylammonium chloride (MAPTAC) 17:83. Furthermore the same study found that oligochitosans without the quaternary ammonium group were inactive in inhibiting coronavirus entry into cells and HTCC was inactive in the viral inhibition of a number of other viruses (e.g. human herpes virus 1, influenza A, adenoviruses and enteroviruses). Additionally, chitosans of molecular weight 5-17 kDa have been shown to be more effective antiviral agents against tobacco mosaic virus than chitosans with a molecular weight of 130 kDa (Davydova, V. N. et al. [Chitosan antiviral activity: dependence on structure and depolymerization method]. Prikl Biokhim Mikrobiol 47, 113-118 (2011)).

New data reported by Meinhardt et al. (“Olfactory transmucosal SARS-CoV-2 invasion as a port of central nervous system entry in individuals with COVID-19. Nat Neurosci, 2020)_showing that SARS-COV-2's neurological symptoms (such as loss of smell and taste, headache, fatigue, nausea and vomiting in more than one-third of individuals and impaired consciousness) is correlated with the entry of SARS-CoV-2 into the brain via the olfactory neurons, due to presence of the virus in the nasal cavity. This means that local interventions, such as with GCPQ that limit viral cell entry in the nasal cavity could have a profound impact on the course and severity of the disease.

Carrageenans (anionic sulphated carbohydrates) have been shown to reduce the duration of disease by 3 days, reduce the number of relapses over a 21 day period by three-fold in influenza and common cold patients, and prevent influenza A viral infections in mice, by preventing viral interaction with relevant cell surface receptors, as disclosed by Koenighofer et aL (“Carrageenan nasal spray in virus confirmed common cold: individual patient data analysis of two randomized controlled trials. Multidisciplinary Resp Med 9, 57, 2014) and Leibbrandt eta., (“Iota-carrageenan is a potent inhibitor of influenza A virus infection”, PLoS One 5, e14320, 2010).

It is thus clear that it is not straightforward in the art to predict which polymers will have activity against viruses in cells and there are various factors which need to be considered to produce polymers which will have activity against viruses in cells, for instance by inhibiting their replication.

### SUMMARY OF THE INVENTION

The current invention is aimed at inhibiting viral infections and/or limiting their severity and is focused on the use of polymers as viral inhibition agents and their use for the treatment or prophylaxis of viral infections.

In accordance with a first aspect of the invention there is provided an amphiphilic carbohydrate compound of molecular weight less than 50 kDa of formula (I) for use in the prevention or treatment of a viral infection:

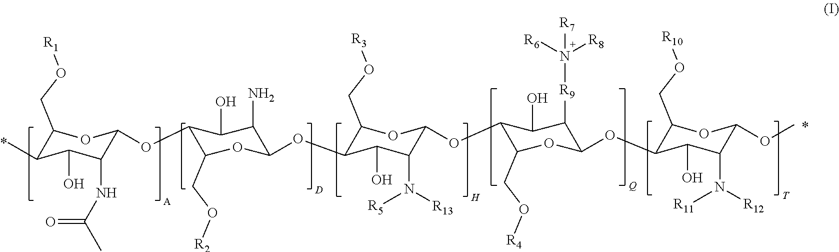

wherein:
the level of unit A is from 0% to 26 mole %;
the level of unit D is from 1% to 95.5 mole %;
the level of unit H is from 1% to 95.5 mole %;
the level of unit Q is from 3% to 40 mole %;
the level of unit T is from 1% to 94.5 mole %;
R₁, R₂, R₃, R₄ and R₁₀ are independently hydrogen or any linear, branched or cyclo form of an alkyl, alkenyl, alkynyl, aryl, acyl group, a sugar substituent selected from glucose, galactose, fructose, and muramic acid, or oligo polyoxa C₁-C₃ alkylene units, optionally substituted with amine, amide or alcohol; wherein at least one of R₁, R₂, R₃, R₄ and R₁₀ is not hydrogen;
R₅ is a hydrophobic, substituted or unsubstituted, linear, branched or cyclo form of a C₄₋₃₀ alkyl, C₄₋₃₀ alkenyl, C₄₋₃₀ alkynyl, C₄₋₃₀ aryl, C₄₋₃₀ amide, C₄₋₃₀ alcohol or C₃₋₃₀ acyl group;
R₆, R₇, and R₈ are independently any linear, branched, or cyclo forms of any alkyl, alkenyl, alkynyl, aryl or acyl group;
R₉ may be present or absent and, when present, is a substituted or unsubstituted alkyl group, a substituted or unsubstituted amine group or a substituted or unsubstituted amide group;
R₁₁ is a substituted or unsubstituted alkyl group, a substituted or unsubstituted ether group or a substituted or unsubstituted alkene group or hydrogen; and
R₁₂ is a substituted or unsubstituted alkyl group, a substituted or unsubstituted ether group or a substituted or unsubstituted alkene group;
R₁₃ is a substituted or unsubstituted alkyl group, a substituted or unsubstituted ether group or a substituted or unsubstituted alkene group or hydrogen;
or a salt thereof.

In accordance with a second aspect of the invention there is provided a pharmaceutical composition for use in the prevention or treatment of a viral infection, comprising one or more pharmaceutically acceptable excipients, and an amphiphilic carbohydrate compound having a molecular weight of less than 50 kDa and is represented by the general formula:

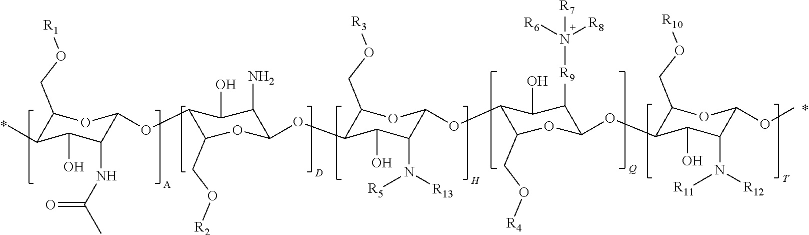

with the groups as defined in the first aspect of the invention.

In accordance with a third aspect of the invention there is provided a method of prevention or treatment of a viral infection wherein a compound or a composition according to the first or second aspect of the invention is administered to the patient, or healthy individual for the purposes of viral prophylaxis.

In this invention we show that derivatised chitosan compounds are effectively able to inhibit viral entry into cells. Due to the structural differences compared to the prior art, one would not have expected the present compounds to confer anti-viral activity.

The presently claimed compounds possess significant advantages for use in viral inhibition and specifically the clinical prevention of viral infections. The compounds are mucoadhesive, chemically stable for at least 18 months and in the case of GCPQ has been subjected to a Good Laboratory Practice toxicology screen, with a no observed adverse effect level defined (Siew, A. et al. Enhanced oral absorption of hydrophobic and hydrophilic drugs using quaternary ammonium palmitoyl glycol chitosan nanoparticles. Molecular Pharmaceutics 9, 14-28, doi:10.1021/mp200469a (2012) and Chooi, K. W. et al. Physical characterisation and long-term stability studies on quaternary ammonium palmitoyl glycol chitosan (GCPQ)—a new drug delivery polymer. J Pharm Sci 103, 2296-2306, doi:10.1002/jps.24026 (2014) and Godfrey, L. et al. Nanoparticulate peptide delivery exclusively to the brain produces tolerance free analgesia. J Control Release 270, 135-144, doi:10.1016/j.jconrel.2017.11.041 (2017)). GCPQ also self assembles into nanoparticles and these nanoparticles may be clustered into microparticles for nasal delivery (Godfrey, L. et al. Nanoparticulate peptide delivery exclusively to the brain produces tolerance free analgesia. J Control Release 270, 135-144, doi:10.1016/j.jconrel.2017.11.041 (2017)). These advantages mean that GCPQ may be given as a nasal spray or by other means for the prevention and treatment of specific viral infections.

U.S. Pat. No. 8,470,371 discloses GCPQ and specifies its use for drug delivery and not as a virus inhibitor.

### BRIEF DESCRIPTION OF THE FIGURES

FIG. 1: Cytotoxicity of GCPQs in vitro. Cell viability was assessed using an XTT assay on Vero E6 cells (A) and A549/ACE2 cells (B). Relative viability of cells (percentage of the untreated control) is shown on y-axis. All assays were performed in triplicate, and average values with standard errors are presented. The letters a to d refer to the GCPQs shown in Table 2.

FIG. 2: Antiviral activity of GCPQs against SARS-CoV-2. Virus replication was evaluated using RT-qPCR. The data are presented as a number of RNA copies per ml of the original sample (top) or as Log Removal Value (LRV) (bottom) compared to untreated samples. The assay was performed in triplicate, and average values with standard errors are presented.

FIG. 3: Replication of SARS-CoV-2 in fully differentiated tissue cultures of the human respiratory epithelium (HAE cultures) in the presence or absence of GCPQ. Virus replication was evaluated using RT-qPCR. The data are presented as a number of viral copies per ml. The assay was performed in triplicate, and median values with range are presented.

FIG. 4: Sagittal SPECT/CT images of radiolabelled GCPQ (10 mg/ kg) at 30 min, 2 h 30 min and 24 hours after nasal administration to male mice (a-c), the nasal delivery device (Naltos device) that may be used to deliver the prophylactic GCPQ powder, permission from Alchemy Pharmatech Ltd.

FIG. 5: Antiviral activity of GCPQs in transgenic mice against SARS-CoV-2 expressing the human ACE2 protein under human cytokeratin 18 promoter (n=6-14). The activity is measured from respiratory tract swabs and brain tissue after application of GCPQ and Remdisivir over a period of 6 days in the presence of a single daily dosage of GCPQ (20 mg/kg), absence of GCPQ (control), and single daily dosage of intramuscular Remdisivir (25 mg/kg). Virus replication was quantified using the RT-qPCR. The data are presented as a number of SARS-CoV-2 copies per ml of the original sample.

### DETAILED DESCRIPTION OF INVENTION

The amphiphilic carbohydrate compound used in this invention is a chitosan derivative. With reference to the formulae in this invention, all percentages refer to mole %. In formula I, it is understood that A+D+H+Q+T will be equal to 100%. It should also be understood that A, D, H, Q and T may form any arrangement in the amphiphilic carbohydrate compound. The arrangement may therefore be entirely random or as a block copolymer form such as ADHQTADHQT etc.

In the invention, A is in the range 0% to 26 mole %. Thus unit A may be absent. If unit A is present, the amphiphilic carbohydrate compound shows a degree of acetylation. Preferably A is in the range 0.5% to 20 mole %, more preferably in the range 0.5% to 15 mole %, even more preferably in the range 0.5% to 10 mole %, even more preferably in the range 0.5% to 5 mole % or 0.5 to 4 mole % or 0.5 to 3 mole %.

In an alternative preferred embodiment, A is in the range 2 to 20 mole %, preferably 2 to mole %, more preferably in the range 2 to 10 mole %, even more preferably in the range 2 to 5 mole % or 2 to 4 mole %.

In an alternative preferred embodiment, A is in the range 1 to 20 mole %, preferably 1 to 15 mole %, more preferably in the range 1 to 10 mole %, even more preferably in the range 1 to 5 mole % or 2 to 5 mole %.

In the invention, D is in the range 1 to 95.5 mole %. In a preferred embodiment of the invention, D is in the range 2% to 94.5 mole %, preferably in the range 10% to 94.5 mole %, more preferably in the range 10% to 90 mole %, typically in the range 20 to 80 mole % or 50% to 75 mole %, more preferably in the range 55% to 75 mole %, even more preferably in the range 65% to 75 mole %.

In the invention, T is in the range 1 to 94.5 mole %. In a preferred embodiment of the invention, T is in the range 2% to 94.5 mole %, preferably in the range 2% to 90 mole %, more preferably in the range 5% to 80 mole %. In a further preferred embodiment, T is in the range 5% to 70 mole %, for instance 5% to 60 mole % or 5% to 50 mole %. In an alternative embodiment, T is in the range 10% to 30 mole %, more preferably in the range 10 to 20 mole % or 20% to 30 mole %.

In the invention, the level of the quaternary ammonium unit, unit Q, is no more than 40mole %. Thus Q is in the range 3% to 40mole %. In a preferred embodiment of the invention, Q is in the range 3% to 30 mole %. It is preferably present in the range 5% to 30 mole %, for instance 5% to 20 mole %, 5% to 15 mole % or 5 to 10 mole %, or for instance 5 to 20 mole %, 10 to 20 mole % or 15 to 20 mole %.

In the invention, H is in the range 1 to 95.5mole %. In a preferred embodiment of the invention, H is in the range 1% to 40 mole %, 1% to 30mole % or 1% to 20 mole %, more preferably in the range 1% to 10 mole %, even more preferably in the range 1% to 5 mole %. In some embodiments, H is in the range 0.5% to 20 mole % or 1% to 20 mole %, for instance, 1 to 10 mole % or 1 to 5 mole %.

The amphiphilic carbohydrate may be in the form of a salt. For instance, the salt can comprise a chloride, iodide, acetate or glucuronide salt.

The molecular weight of the amphiphilic carbohydrate compound has a molecular weight of less than 50 kDa. It is preferably in the range 1-40 kDa or 1-30 kDa, for instance 5-30 kDa or 10-30 kDa. Molecular weight is preferably measured using Gel-permeation chromatography—multi-angle light scattering (GPC-MALLS). The molecular weight will be the mean average measurement for all the polymer chains present in a sample.

The amphiphilic carbohydrate compound is capable of self-assembly into nanoparticles in aqueous media.

R₁, R₂, R₃, R₄ and R₁₀ are independently hydrogen or any linear, branched or cyclo form of an alkyl, alkenyl, alkynyl, aryl, acyl group, a sugar substituent selected from glucose, galactose, fructose, and muramic acid, or oligo polyoxa C₁-C₃ alkylene units, optionally substituted with amine, amide or alcohol. Preferably these groups are independently selected from hydrogen, a substituted or unsubstituted alkyl group, a substituted or unsubstituted ether group, or a substituted or unsubstituted alkene group.

Typically, R₁, R₂, R₃, R₄ and R₁₀ may be C₁-C₄ linear alkyl groups. Typically, R₁, R₂, R₃, R₄ and R₁₀ may be C₁-C₄ linear glycol-based groups. Typically, R₁, R₂, R₃, R₄ and R₁₀ are any of the following sugar substituents: glucose, galactose, fructose, and muramic acid. R₁, R₂, R₃, R₄ and R₁₀ may be oligo polyoxa C₁-C₃ alkylene units such as ethylene glycol oligomers. All of R₁, R₂, R₃, R₄ and R₁₀ may be CH₂CH₂OH.

R₁, R₂, R₃, R₄ and R₁₀ may also be hydrophilic.

Typically, R₅ is a hydrophobic, substituted or unsubstituted, linear, branched or cyclo form of a C₄₋₃₀alkyl, C₄₋₃₀alkenyl, C₄₋₃₀alkynyl, C₄₋₃₀aryl, amine, C₄₋₃₀amide, C₄₋₃₀alcohol or C₃₋₃₀ acyl group. The group R₅ is preferably selected from a substituted or unsubstituted group which is an acyl group such as a C₃₋₃₀acyl group, an alkyl group such as a C₄₋₃₀alkyl group, an alkenyl group such as a C₄₋₃₀ alkenyl group, an alkynyl group such as a C₄₋₃₀alkynyl group, an aryl group such as a C₅₋₂₀aryl group, a multicyclic hydrophobic group with more than one C₄-C₈ ring structure such as a sterol (e.g. cholesterol), a multicyclic hydrophobic group with more than one Ca-Co heteroatom ring structure, a polyoxa C₁-C₄ alkylene group such as polyoxa butylene polymer, or a hydrophobic polymeric substituent such as a poly (lactic acid) group, a poly(lactide-co-glycolide) group or a poly(glycolic acid) group. The R₅ group may be linear, branched or cyclo groups.

Preferred examples of R₅ groups include those represented by the formulae CH₃(CH₂)ₙ—CO— or CH₃(CH₂)ₙ— or the alkeneoic acid CH₃(CH₂)ₚ—CH═CH—(CH₂)_{q}—CO—, where n is between 4 and 30, and more preferably between 6 and 20, and p and q may be the same or different and are between 4 and 16, and more preferably are between 4 and 14. A particularly preferred class of R₅ substituents are linked to the chitosan monomer unit via an amide group (including the pendant NH in the formula), for example as represented by the formula CH₃(CH₂)ₙCO—, where n is between 2 and 28. Examples of amide groups are produced by the coupling of carboxylic acids to the amine group of chitosan. Preferred examples are fatty acid derivatives CH₃(CH₂)ₙCOOH such as those based on capric acid (n=8), lauric acid (n=10), myristic acid (n=12), palmitic acid (n=14), stearic acid (n=16) or arachidic acid (n=18).

R₆, R₇, and R₈ are independently any linear, branched, or cyclo forms of any alkyl, alkenyl, alkynyl, aryl or acyl group. R₆, R₇ and R₈ are preferably independently selected from a substituted or unsubstituted alkyl group such as a C₁₋₁₀ alkyl group. R₆, R₇ and/or R₈ may be linear or branched. Preferably, R₆, R₇ and R₈ are independently selected from methyl, ethyl or propyl groups and are preferably methyl groups. Conveniently, R₆, R₇ and R₈ form a quaternary ammonium group which is hydrophilic. Hydrophilic groups are groups which are well hydrated by water and associate on a molecular level with water.

The R₉ group may be present or absent in the general formula. R₉ may be present or absent and, when present, is a substituted or unsubstituted alkyl group, a substituted or unsubstituted amine group or a substituted or unsubstituted amide group. Generally, R₉ is not a 2-hydroxypropyl group. When absent, it provides a quaternary ammonium functional group that is directly linked to the monomer unit of the chitosan backbone. When the R₉ group is present it may be a unsubstituted or substituted alkyl group (e.g. a C₁₋₁₀ alkyl group but generally not a 2-hydroxypropyl group —CH₂—CH(OH)—CH₂—) for example as represented by —(CH₂)ₙ— wherein n is preferably 1 to 4. A preferred example of the R₉N+R₆R₇R₈ substituent is provided by coupling betaine (—OOC—CH₂—N⁺—(CH₃)₃) to the amine of the D unit providing an amide group such as in: —NH—CO—CH₂—N+R₆R₇R₈.

R₁₁ is a substituted or unsubstituted alkyl group, a substituted or unsubstituted ether group or a substituted or unsubstituted alkene group or hydrogen. Preferably R₁₁ is selected from hydrogen and a substituted or unsubstituted alkyl group such as a C₁₋₁₀ alkyl group. R₁₁ may be linear or branched. Preferably, R₁₁ is selected from methyl, ethyl or propyl groups and is preferably a methyl group. Alternatively, it is an OH— substituted alkyl group, preferably of formula CH₂CH₂OH.

R₁₂ is a substituted or unsubstituted alkyl group, a substituted or unsubstituted ether group or a substituted or unsubstituted alkene group. Preferably R₁₂ is selected from substituted or unsubstituted alkyl group such as a C₁₋₁₀alkyl group. R₁₂ may be linear or branched. Preferably, R₁₂ is selected from methyl, ethyl or propyl groups. Alternatively, it is an OH-substituted alkyl group, preferably of formula CH₂CH₂OH. Typically, R₁₂ is a C₁₋₁₀ alkyl group. R₁₂ may be linear or branched. Preferably, R₁₂ is selected from methyl, ethyl or propyl groups and is preferably a methyl group.

R₁₃ is a substituted or unsubstituted alkyl group, a substituted or unsubstituted ether group or a substituted or unsubstituted alkene group or hydrogen. Preferably R₁₃ is selected from hydrogen and a substituted or unsubstituted alkyl group such as a C₁₋₁₀ alkyl group. R₁₃ may be linear or branched. Preferably, R₁₃ is selected from methyl, ethyl or propyl groups, most preferably a methyl group. Alternatively, it is an OH-substituted alkyl group, preferably of formula CH₂CH₂OH. Most preferably R₁₃ is hydrogen.

The total number of monomer units of A+D+H+Q+T may be about 5 to 100. The total number of monomer units of A+D+H+Q+T may be less than about 220.

In a preferred embodiment of the invention, the amphiphilic carbohydrate compound is a form of N-palmitoyl,N-monomethyl,N,N-dimethyl,N,N,N-trimethyl-6-O-glycolchitosan (GCPQ). This is known to be an amorphous compound (Godfrey et al., “Nanoparticulate peptide delivery exclusively to the brain produces tolerance free analgesia”, J. Control Release 2017, 270, 135-144).

As indicated, some of the substituents described herein may be either unsubstituted or substituted with one or more additional substituents as is well known to those skilled in the art. Examples of common substituents include halo; hydroxyl; ether (e.g., C₁₋₇ alkoxy); formyl; acyl (e.g. C₁₋₇ alkylacyl, C₅₋₂₀ arylacyl); acylhalide; carboxy; ester; acyloxy; amido; acylamido; thioamido; tetrazolyl; amino; nitro; nitroso; azido; cyano; isocyano; cyanato; isocyanato; thiocyano; isothiocyano; sulfhydryl; thioether (e.g., C₁₋₇ alkylthio); sulphonic acid; sulfonate; sulphone; sulfonyloxy; sulfinyloxy; sulfamino; sulfonamino; sulfinamino; sulfamyl; sulfonamido; C₁₋₇ alkyl (including, e.g., unsubstituted C₁₋₇ alkyl, C₁₋₇ haloalkyl, C₁₋₇ hydroxyalkyl, C₁₋₇ carboxyalkyl, C₁₋₇ aminoalkyl, C₅₋₂₀ aryl-C₁₋₇ alkyl); C₃₋₂₀ heterocyclyl; and C₅₋₂₀ aryl (including, e.g., C₅₋₂₀ carboaryl, C₅₋₂₀ heteroaryl, C₁₋₇ alkyl-C₅₋₂₀ aryl and C₅₋₂₀ haloaryl) groups.

The term “ring structure” as used herein, pertains to a closed ring of from 3 to 10 covalently linked atoms, yet more preferably 3 to 8 covalently linked atoms, yet more preferably 5 to 6 covalently linked atoms. A ring may be an alicyclic ring, or aromatic ring. The term “alicyclic ring,” as used herein, pertains to a ring which is not an aromatic ring.

The term “carbocyclic ring”, as used herein, pertains to a ring wherein all of the ring atoms are carbon atoms.

The term “carboaromatic ring”, as used herein, pertains to an aromatic ring wherein all of the ring atoms are carbon atoms.

The term “heterocyclic ring”, as used herein, pertains to a ring wherein at least one of the ring atoms is a multivalent ring heteroatom, for example, nitrogen, phosphorus, silicon, oxygen or sulphur, though more commonly nitrogen, oxygen, or sulphur. Preferably, the heterocyclic ring has from 1 to 4 heteroatoms.

The above rings may be part of a “multicyclic group”.

Preferably, the amphiphilic carbohydrate compound is N-palmitoyl-N-monomethyl-N,N-dimethyl-N,N,N-trimethyl-6-O-glycolchitosan, otherwise known as quaternary ammonium palmitoyl glycol chitosan (GCPQ). In this compound, R₆, R₇ and R₈ are methyl; R₁, R₂, R₃, R₄ and R₁₀ are —CH₂CH₂OH; R₉ is absent; R₅ is —C(═O)—C₁₅H₃₁ and R₁₃ is hydrogen. R₁₁ and R₁₂ are either hydrogen or methyl and both may not be hydrogen.

In this case, the palmitoylation level (corresponding to group H) is preferably between 5-50% per monomer, for instance, 10-20% per monomer. The quaternisation level (Q) is preferably between 3-40% per monomer, preferably 10-30% per monomer. The molecular weight of the GCPQ is typically in the range 1-40kDa or 1-30 kDa, for instance 5-30 kDa or 10-30 kDa.

The amphiphilic carbohydrate compound is capable of self-assembling into particles in aqueous media without the presence of other agents such as tripolyphosphate. Generally, micelles are formed.

The amphiphilic carbohydrate may form particulate aggregates. These may be formed by the aggregation of individual amphiphile molecules and have a mean particle size of between 10 nm and 20 μm. The mean particle size can readily be determined microscopically or by using photon correlation spectroscopy and is conveniently determined in aqueous dispersions prior to filtration. More preferably, the polymeric micellar aggregates have a minimum mean particle size of at least 10 nm, and more preferably at least 30 nm, and a maximum mean particle size which is preferably 10 μm or less.

In the invention, the amphiphilic carbohydrate compound may be used alone or formulated with one or more drugs. The drug may be a hydrophobic drug. The amphiphilic carbohydrate compound is capable of self-assembly into nanoparticles in aqueous media. Pharmaceutical compositions of the present invention may comprise nanodispersions of the nanoparticles of the amphiphilic carbohydrate compound.

The amphiphilic carbohydrate compound of the invention is useful against viral infections per se. Therefore, it may be used alone in the treatment or prevention of a viral infection.

Thus the amphiphilic carbohydrate compound is itself an active agent. In a pharmaceutical composition of the invention, it may be the only active agent/ingredient, i.e. there may be no further drugs present. For instance, a pharmaceutical composition may consist essentially only of the amphiphilic carbohydrate compound and one or more pharmaceutically acceptable excipients.

In an embodiment of the invention, the amphiphilic carbohydrate compound forms nanoparticles which can be loaded with drug. The drug is typically encapsulated by the self-assembled positively charged amphiphilic polymers. A dispersion of carbohydrate and drug may be formed which is clear or translucent. Generally the amphiphilic compound is mixed with drug and a dispersion is prepared by vortexing and probe sonicating the mixture or by high-pressure homogenisation of the mixture. Typically, the drug is a hydrophobic drug. A hydrophobic drug is one which is poorly soluble in aqueous media, such as water. By poorly soluble drugs is meant where one gram of a drug requires more than 10,000 ml of solvent (water) to be solubilised. Alternatively, this means a drug which has a solubility of less than 0.1 mgmL⁻¹ in water.

The hydrophobic drug is typically encapsulated by the amphiphilic carbohydrate compound. The hydrophobic drug may be an analgesic, antibiotic, anticoagulant, antidepressant, anticarcinogen, anticarcinoma, anti-inflammatory, antihistamine, antiemetic, anxiolytic, anticonvulsive, antipsychotic, antipyretic, antiviral, antidiabetic, sedative, antihypertensive or a cardiovascular drug.

The hydrophobic drug may act as a diuretic or antidiuretic, chronotrope, inotrope, decongestant, bronchodilator, anticholinergic, antithrombotic, antimicrobial or antifungal.

When a drug is present, it may be an anti-viral drug, which further potentiates the action of the amphipilic carbohydrate compound. Typically, the drug is effective against respiratory viruses.

The amphiphilic carbohydrate compound of the invention is effective as an anti-viral agent. The virus may be for instance selected from coronaviruses, influenza and ebola, herpes viruses, adenoviruses and enteroviruses. For instance, the compound may be effective against human herpes virus 1 or influenza A.

Preferably, the amphiphilic carbohydrate compound is used in the prevention and/or treatment of infections caused by alphacoronaviruses or betacoronaviruses. The coronavirus may be severe acute respiratory syndrome coronavirus 2 (SARS-CoV-2), otherwise known as Covid-19.

The amphiphilic carbohydrate compound of the invention may be effective via inhibition of viral entry into cells.

Polymers of the invention may be useful in the treatment and prevention of respiratory infections manifested by insufficiency and impairment of respiratory functions in humans or animals, for instance in the treatment and prevention of infections of the upper respiratory tract, lower respiratory tract infections, croup in children, gastrointestinal infections, infections of the nervous system, and Kawasaki disease.

In the compositions of the invention the drug, if present with the amphiphilic carbohydrate, is preferably present at a concentration in the range 0.001-10% w/v. When concentrations are expressed in % w/v, this means the amount of solid, in g, contained in 100 mL or 100 g of composition. Typically, the ratio of amphiphilic carbohydrate compound to drug (if present) is within the range of 1:1 to 50:1, more preferably 1:1 to 20:1.

Typically, the ratio of amphiphilic carbohydrate compound to drug to pharmaceutically acceptable carrier may be about 1-40 mg:1 mg:1 g, for instance 1-5 mg:1 mg:1 g.

The pharmaceutical composition may be in the form of any of the following: tablets, suppositories, liquid capsule, powder form, or a form suitable for pulmonary or nasal delivery.

When tablets are used for oral administration, typically used carriers include sucrose, lactose, mannitol, maltitol, dextran, corn starch, typical lubricants such as magnesium stearate, preservatives such as paraben, sorbin, anti-oxidants such as ascorbic acid, α-tocopheral, cysteine, disintegrators or binders. When administered orally as capsules, effective diluents include lactose and dry corn starch. A liquid for oral use includes syrup, suspension, solution and emulsion, which may contain a typical inert diluent used in this field, such as water, in addition, sweeteners or flavours may be contained. Suppositories may be prepared by admixing the compounds of the present invention with a suitable non-irritative excipient such as those that are solid at normal temperature but become liquid at the temperature in the intestine and melt in the rectum to release the active ingredient, such as cocoa butter and polyethylene glycols.

Additional ingredients that may be included in the formulation include tonicity enhancers, preservatives, solubilisers, non-toxic excipients, demulcents, sequestering agents, pH adjusting agents, co-solvents and viscosity building agents.

Tonicity is adjusted if needed typically by tonicity enhancing agents. Such agents may, for example be of ionic and/or non-ionic type. Examples of ionic tonicity enhancers are, but are not limited to, alkali metal or earth metal halides, such as, for example, CaCl2, KBr, KCI, LiCI, Nal, NaBr or NaCl, Na2SO4 or boric acid. Non-ionic tonicity enhancing agents are, for example, urea, glycerol, sorbitol, mannitol, propylene glycol, or dextrose. These agents may also serve as the active agents in certain embodiments.

For the adjustment of the pH, preferably to a physiological pH, buffers may be especially useful. The pH of the present solutions should be maintained within the range of 5.5 to 8.5, The challenge is that for ionic complexation to occur may require a more acidic or alkaline pH. Suitable buffers may be added, such as, but not limited to, boric acid, sodium borate, potassium citrate, citric acid, sodium bicarbonate, TRIS, and various mixed phosphate buffers (including combinations of Na₂HPO₄, NaH₂PO₄ and KH₂PO₄) and mixtures thereof. Generally, buffers will be used in amounts ranging from about 0.05 to 2.5 percent by weight

The pharmaceutical composition may be formulated for administration by any route, for instance, oropharyngeal, oral, parenteral, nasal, by inhalation, topical ocular or topical. The drug is preferably delivered to the human or animal body by intranasal or oropharyngeal delivery. The formulation may be a powder or liquid dispersion. Formulation of the pharmaceutical composition into a nasal and oropharyngeal spray for intranasal or oropharyngeal delivery is particularly preferred. The delivery of the nasal and/or oropharyngeal spray may be accomplished by a spay device such as a Naltos™ nasal delivery device (Alchemy Pharmatech).

The dose can be determined on age, body weight, administration time, administration method, combination of drugs, the severity of the clinical condition or the actual condition for which a patient is undergoing therapy and other factors. While the daily doses may vary depending on the conditions and body weight of patients, the species or active ingredient, and administration route, in the case of oral use, the daily doses may be about 0.1mg-2 g/person/day, or from 0.5-1000 mg/person/day or from 5-500 mg/person/day or from 10-250 mg/person/day or from 25-200 mg/person/day.

The current invention is aimed at inhibiting viral infections and/ or limiting their severity and is focused on the use of polymers as viral inhibition agents and their use for the treatment or prophylaxis of viral infections. The compositions of the present invention may reduce viral replication such that the titre of virus measured post treatment as compared to pre-treatment is at least twofold or threefold or fourfold or fivefold or tenfold or one hundredfold less. The compositions of the present invention may reduce viral replication such that the titre of virus measured post treatment as compared to pre-treatment is between 10 to 90% or 10 to 75% or 10 to 50% or 10 to 25% that of the pre-treatment titre.

### EXAMPLES

### Example 1

### Materials

a. Cell lines: Vero E6 (ATCC: CRL-1586), A549 with the ACE2 overexpression (A549/ACE2+; prepared in our laboratory using ATCC CCL-185 cell line);
b. Virus: SARS-CoV2 isolate 026V-03883 was used for the in vitro work; Charité—Universitätsmedizin Berlin, Germany, European Virus Archive—Global (EVAg); https://www.european-virus-archive.com; SARS-CoV-2 isolate (Munchen-1.2 2020/984) was used for the in vivo work.
c. Medium: DMEM—Dulbecco's Modified Eagle Medium (Thermo Fisher Scientific, Poland) supplemented with 2-3% heat-inactivated Foetal Bovine Serum (FBS) (Thermo Fisher Scientific, Poland), penicillin (100 U/mL) (Thermo Fisher Scientific, Poland) and streptomycin (100 μg/mL) (Thermo Fisher Scientific, Poland), and ciproflocaxin (5 μg/mL);
d. Cultures: MucilAir™ Human Airway Epithelial (HAE) cultures were used for the ex vivo analysis (Epithelix Sarl, Switzerland). All cultured were carried out at 37° C. under 5% CO₂; e. Animals: Transgenic mice expressing human ACE2 protein under cytokeratin 18 promoter (Jackson laboratory, USA)
f. XTT cell viability kit (Biological Industries, Israel)
g. Viral DNA/RNA isolation kit (A&A Biotechnology, Poland)
h. High-capacity cDNA reverse transcription kit (Thermo Fisher Scientific, Poland)
i. Real-time qPCR kit (RT-HS-PCR mix probe, A&A Biotechnology, Poland)
j. Real-time qPCR oligonucleotides are listed in Table 1.

All experiments with the infectious agents were carried out in the ABSL3+ facility approved for work with the airborne BSL3 pathogens, including the SARS-CoV-2 virus.

TABLE 1
Real-time qPCR oligonucleotides.

| Oligonucleotides | Sequence (5′ −> 3′) |
|---|---|
| 5′ primer (Forward) | CACATTGGCACCCGCAATC |
| 3′ primer (Reverse) | GAGGAACGAGAAGAGGCTTG |
| Fluorescent probe | 6-ACTTCCTCAAGGAACAACATTGCCA- |
| | BHQ-1 |

### i. GCPQ Compounds

GCPQs used in the experiments are listed in Table 2. The compounds were resuspended in 1×PBS to the final concentration of 5 mg/mL. All stocks were stored at 4° C.

TABLE 2
GCPQs

| GCPQa | GCPQc |
|---|---|
| Molecular Weight = 10 kDa | Molecular Weight = 15 kDa |
| Mole % palmitoyl groups = 18 | Mole % palmitoyl groups = 18 |
| Mole % quaternary ammonium | Mole % quaternary ammonium |
| groups = 18 | groups = 20 |
| GCPQb | GCPQd |
| Molecular Weight = 30 kDa | Molecular Weight = 60 kDa |
| Mole % palmitoyl groups = 19 | Mole % palmitoyl groups = 18 |
| Mole % quaternary ammonium | Mole % quaternary ammonium |
| groups = 19 | groups = 16 |

### METHODS

### Tissue Culture

a) The cytotoxicity of compounds was assessed by incubating confluent monolayers of Vero E6 and A549/ACE2+ cells with a range of GCPQ compound concentrations. The XTT assay was carried out 48 hours later, according to the manufacturer's protocol using 200,000 cells per well and DMEM supplemented with foetal bovine serum, penicillin and streptomycin (please see above under materials).
b) The ability of each compound to inhibit the virus replication was determined by infecting confluent Vero E6 and A549/ACE2+ monolayers with 400 TCID50/mL of the SARS-CoV-2 virus in the presence of test compounds or PBS (TCID50=Median Tissue Culture Infectious Dose). Mock controls (cell lysate without the virus) and medium (supplemented DMEM—please see above) controls were included. Each compound was present during and after the infection. The cells were then incubated for 2 hours at 37° C. and 5% CO₂. Afterward, the cells were washed three times with PBS, and each compound was re-applied into the cell monolayer. 100 μL of the cell culture supernatants were subsequently collected from each designated well after two days of culture at 37° C. and in a 5% CO₂ environment. The experiments were carried out in triplicate.
c) Virus replication inhibition in HAE was evaluated by infecting MucilAir™ with SARS-CoV-2 isolate 026V-03883 (in vitro) virus at 5000 TCID50/mL in the presence of GCPQa or PBS. GCPQa diluted in PBS was added to the apical side of the insert (200 μg/ml or 500 μg/ml) and incubated at 37° C. for 30 minutes before the infection. After the pre-incubation was completed, the compound was removed and fresh dilutions of the compound with the virus were added and incubated for 2 hours at 37° C. Next, the apical side of the HAE was washed thrice with PBS and each compound was re-applied and incubated again for 30 minutes at 37° C. After the last incubation with the GCPQ, the samples (50 μL) were collected and the HAE cultures were left in air-liquid interphase. Every 24 hours the HAE apical surface was incubated for 30 minutes with the GCPQ or PBS and the samples were collected for virus yield evaluation.
d) Viral RNA was isolated from the cell culture supernatants and reverse-transcribed into cDNA, which was subsequently used as a template for the real-time qPCR reaction.

An aliquot of the cell culture supernatant (100 μL) was collected from each sample of each experiment and directly inactivated by adding lysis buffer (400 μL, R9F buffer, Viral DNA/RNA Kit). cDNA samples were prepared with a High Capacity cDNA Reverse Transcription Kit according to the manufacturer's instructions. Briefly, 5 μL of isolated RNA was mixed with 2× concentrated RT-PCR mix and incubated as follows: 10 min at 25° C., 2 hrs at 37° C., 5 min at 85° C. cDNA was stored at −20° C. until use. Viral RNA yield was assessed using real-time PCR (7500 Fast Real-Time PCR; Life Technologies, Poland). cDNA was amplified in a reaction mixture containing 1× qPCR Master Mix (A&A Biotechnology, Poland), in the presence of probe (100 nM) and primers (450 nM each), sequences provided in Table 1.

The reaction was carried out according to the scheme: 2 min at 50° C. and 10 min at 92° C., followed by 40 cycles of 15 s at 92° C. and 1 min at 60° C. In order to assess the copy number for N gene, DNA standards were prepared, as described before (Milewska, A. et al. Replication of Severe Acute Respiratory Syndrome Coronavirus 2 in Human Respiratory Epithelium. J Virol 94, doi:10.1128/JVI.00957-20 (2020)).

The number of viral RNA copies/mL was calculated by comparing the value obtained for each well with that of serial dilutions of samples containing a known number of cDNA copies/mL (standards). The difference in viral yield was also analysed as the log removal value (LRV), showing the relative decrease in the amount of virus in cell culture media compared to the control.

### Intranasal Delivery in a Healthy Animal Model

GCPQ (molecular weight=10 kDa, mole % palmitoyl groups=16 and mole % quaternary ammonium groups=13) was radiolabelled using a two stage strategy: first an acylating reagent (N-succinimidyl-3[4-hydroxyphenyl]propionate—the Bolton and Hunter reagent (BH)) was initially covalently coupled to GCPQ and then the GCPQ-BH complex was iodinated with 1251. Briefly, GCPQ (90 mg) was dissolved in DMSO (3 mL). To this solution was added 200 μL of triethylamine and 0.05 molar equivalents (10 mg) of BH reagent and the reaction allowed to proceed overnight at room temperature with stirring. The next day, the GCPQ-BH conjugate was precipitated using an acetone: diethyl ether mixture (1:2, v/v) and the pellet was washed 3 times with the same acetone: diethyl ether mixture. The washed pellet was dissolved in methanol (2 mL) and dialyzed against water overnight. The dialysed GCPQ-BH was then freeze dried and collected. Labelling of GCPQ-BH with ¹²⁵I was performed using iodination beads® (Thermo Scientific Pierce, UK). Briefly, GCPQ-BH (20 mg) and 100 mg GCPQ were dissolved in methanol with stirring, then the methanol was removed under vacuum and Tris-HCL buffer (25 mM, pH 4.8, 1.8 mL) was added to the dry film to produce a final concentration of 66.7 mg/mL. This solution was then added to a tube containing the 1125 (1 mCi, 17 Ci/mg, 0.392 nmol, Perkin-Elmer, USA) and four iodination beads® (Thermo Scientific Pierce, UK). The reaction was incubated for 1.5 hours at room temperature, after which the reaction was terminated by separating the solution from beads. PD Spin Trap G-25 Columns (GE Healthcare Life Sciences, UK), that are prepared by vortexing and discarding of the eluting storage buffer by centrifuging (2800 rpm for 1 min), were used in order to remove the free iodine (with the free iodine removed through the addition of 50 μL of the reaction per column and centrifuging at 2,800 rpm for 2 min). The eluent was placed in Amicon ultra centrifugal filters (3 kDa, Millipore, USA) with 200 pL H₂O, and was subject to repeated washes (through centrifuging at 10,000 rpm for 10 min), until the washed out water produced negligible counts.

All animal experiments were performed under a UK Home Office licence (PPL 70/8224) and were approved by the local ethics committee—the UCL Animal Welfare and Ethical Review Body. The animal experiments were carried out in accordance with the guidelines contained in the licence, and ARRIVE guidelines were followed, however there was no blinding or randomisation carried out. An exploratory study on a Balb/C Male mouse weighing between 25-30 g (Charles River, UK), which was allowed free access to standard rodent chow and water was carried out. Radiolabelled GCPQ-BH was intranasally administered (10 mg/kg, 1.2 MBq) using a pipette to place 5 μL of the radiolabelled material into the mouse nares and allowing the mouse to sniff in the dose. At various time points after the administration of the radiolabelled GCPQ, animals were anaesthetised using isofluorane (1-2% v/v in oxygen), maintained at 37° C. and submitted for NanoSPECT/CT analysis (Mediso, USA).

### In Vivo SPECT/CT Imaging and Analysis

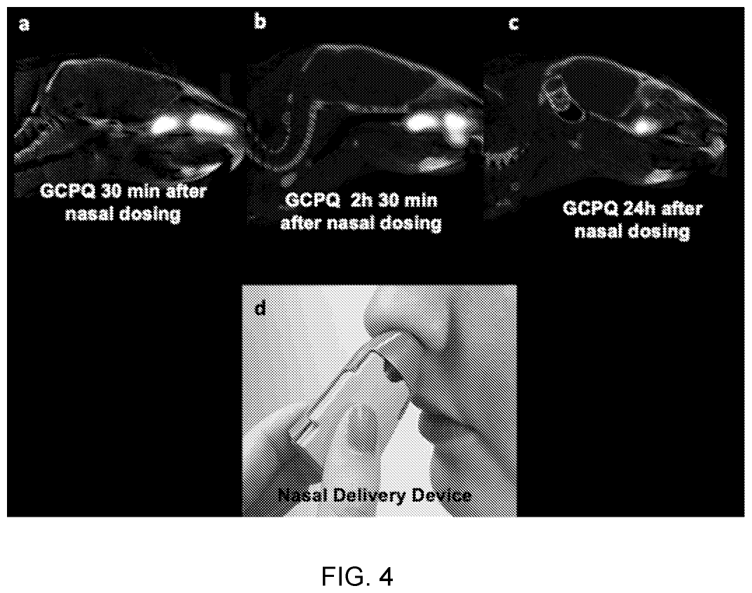

SPECT/CT scans of the mouse head at 30 min, 2 h 30 min and 24 h after nasal administration were acquired (FIG. 4) using a NanoSPECT/CT scanner (Mediso, Hungary). SPECT images were obtained over 30 minutes using a 4-head scanner with nine 1.4 mm pinhole apertures in helical scan mode with a time per view of 60 seconds. CT images were subsequently acquired using a 45 kilo volt peak (kVp) X-ray source, 500 ms exposure time in 180 projections, a pitch of 0.5 with an acquisition time of 4:30 20 minutes. Body temperature was maintained by a warm air blower and the respiration and core body temperature was monitored throughout. CT images were reconstructed using Bioscan InVivoScope (Bioscan, USA) software in voxel size 124×124×124 μm, whereas SPECT images were reconstructed using HiSPECT (ScivisGmbH, Bioscan) in a 256×256 matrix. Images were fused and analysed using VivoQuant (Invicro, A Konica Minolta Company) software. 3D Regions of Interest (ROIs) were created for the uptake within the nares for each time point and the activity calculated as the percentage of the administered dose. Representative images are scaled the same (same min and max). After the final scan the mouse was sacrificed and the entire head of the mouse analysed using a curimeter (Capintech, Mirion Technologies, UK) for ex vivo validation of ¹²⁵I concentration.

### In Vivo Viral Inhibition in Transgenic Mice Expressing ACE2 Receptor

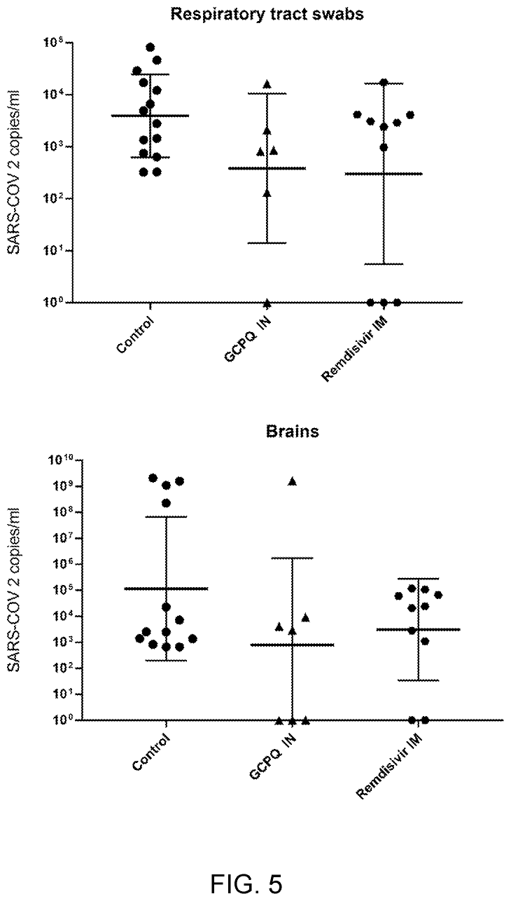

Transgenic mice expressing the human ACE2 protein under the human cytokeratin 18 promoter were experimented for antiviral activity against SARS-CoV-2 (Munchen-1.2 2020/984). There were 3_experimental groups, consisting of; GCPQ administration, a control (no GCPQ), and a Remdisivir group, each consisting of 10 animals, except the control group which had 14. The mice were quarantined for at least 7 days prior to the experiment. GPCQa was administered once daily intranasally (20 mg/kg per day). The treatment control group received remdesivir intramuscularly (25 mg/kg per day). The timeline of the experiments were 7 days, starting from day −1 until day 6 post-infection, during which the mice were intranasally administered with GCPQ or Remdisivir and examined and weighed every 24 hours. Mice were provided with free and permanent access to water during each experiment for the entire length. On day 0 the mice were infected intranasally with the SARS-CoV-2 virus (Munchen-1.2 2020/984; 5 μl to each nostril) at 426,000 TC1D₅₀/ml, which corresponded to ˜3×105 pfu. The virus was then propagated and titrated on Vero cells. On day 6 post-infection, animals were sacrificed under anaesthesia and the lungs and brain tissues extracted. These tissues were homogenised using a bead homogeniser and nasal swabs were also taken. The Viral RNA was isolated using mirVana™ miRNA Isolation kit (ThermoFisher Scientific, Poland), and the viral infection quantified using the RT-qPCR. FIG. 5 shows the antiviral activity in the brains and from the respiratory tract swabs following nasal dosing of GCPQ and Remdesivir.

### Statistical Analyses

Statistical analyses were carried out using one way ANOVA plus Tukey's post hoc tests. Statistical significance was set at a p<0.05.

### Results

### Cytotoxicity

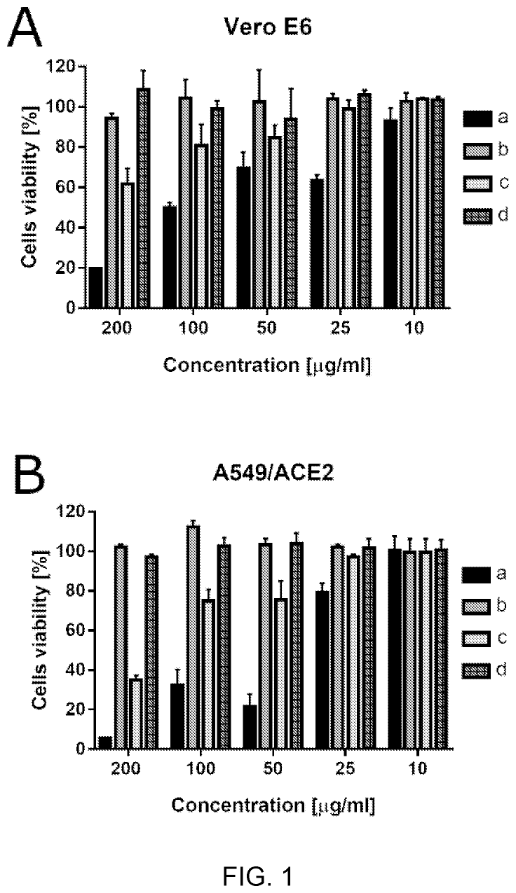

Four GCPQ polymers (see Table 2) were tested. First, the cytotoxicity of polymers was analysed on Vero E6 and A549/ACE2+ cells. The results of the analysis are shown in FIG. 1.

The analysis revealed different toxicities for the different GCPQs. For the virus assays, only non-toxic concentrations were tested: 10 μg/ml of GCPQa, 25 μg/ml for GCPQc, and 200 μg/ml for GCPQb and GCPQd.

### Antiviral Activity

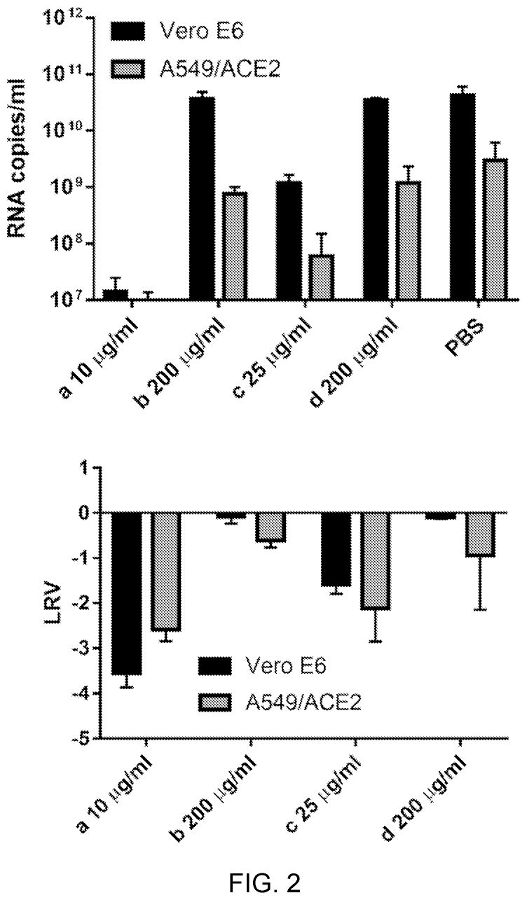

The antiviral activity of GCPQs was analysed on Vero E6 and A549/ACE2+ cells. Each analysis was performed in triplicate. The results of the experiment are presented in FIG. 2.

The analysis demonstrated that the presence of chitosans GCPQa and GCPQc at non-toxic concentrations significantly hamper SARS-CoV-2 replication in vitro. GCPQa and GCPQc showed anti-coronaviral activity at non-toxic concentrations. GCPQa showed the highest toxicity and anti-SARS-CoV-2 potential (−3 to 4 logs decrease in viral load at 10 μg/mL).

### Antiviral Activity in Human Airway Epithelial (HAE) Cells

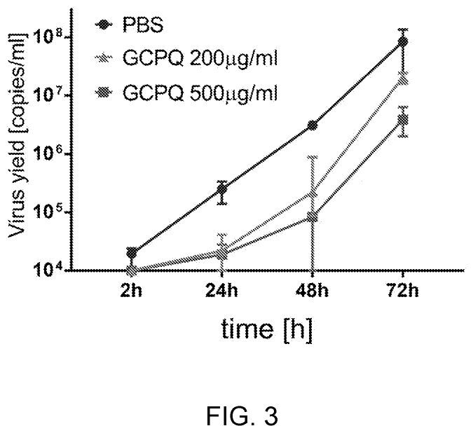

To validate our observation on the anti-SARS-CoV-2 activity of GCPQa, fully differentiated HAE ex vivo model was used, reconstituting ex vivo the human respiratory epithelium. Two different concentration of GCPQa were evaluated (200 μg/ml and 500 μg/ml) and PBS was used as a control. Each analysis was performed in triplicate and the results are shown in FIG. 3. At the 48 and 72 hour time points, GCPQa at 500 μg/mL is statistically significantly different from the PBS treated samples (p<0.05).

The results show that chitosan GCPQa hampers SARS-CoV-2 replication in the HAE ex vivo model. A lower viral yield was detected in the cultures treated with GCPQ than in the control with PBS after 72 h of infection.

### Nasal Delivery

The intranasal delivery of GCPQ to male mice resulted in the polymer being detectable in the nares of mice up to 24 hours after dosing as would be expected since the polymer is mucoadhesive. 3D ROls of the GCPQ uptake on SPECT images in FIG. 4 indicated that directly after administration (30 min) 28.22% of the administered dose was found to be within the nares, after 2h 30 min this had reduced slightly to 25.13% of the administered dose and at 24 h, 13.13% of the administered dose was found to be retained in the nares. Ex vivo curimeter analysis of the mouse head at 24 h, also confirmed that 13.5% of the administered dose was found in the nares 24 hours after dosing, which is slightly higher than that obtained by SPECT, but is reflective of the radioactivity within the whole head and not just the nares area.

### In Vivo Viral Inhibition

The initial viral titre used in these experiments was significantly higher than contained in human infective influenza breath samples (3×105 pfu in this study vs influenza qPCR RNA copy numbers of 3.8×104 in a 30 minute fine aerosol breath sample and 1.2×104 in a 30 minutes coarse aerosol breath sample according to Yan et aL (Yan, J. et al.

Infectious virus in exhaled breath of symptomatic seasonal influenza cases from a college community. Proceed Natl Acad Sci 115, 1081, doi:10.1073/pnas.1716561115 (2018).

However, there was inhibition of viral replication in the mouse nasal passages and brains at the once daily GCPQ dose (FIG. 5).

The assay according to FIG. 5 showed an inhibition of SARS-CoV2 replication in the presence of GCPQ at non-toxic concentrations in both the respiratory tract swabs and brains relative to the control. Relative to the Remdisivir, the average number of RNA copies with GCPQ in the respiratory tract swabs was almost level, but overall gave slightly less inhibition. Further, in the brains assay, the average inhibition with GCPQ was greater than the Remdisivir, however the error bars were larger owing to a single result with high SARS-CoV-2 copies/ml.

The high initial viral titre (comparatively higher than contained in a human infective droplet) in this model means that it is more difficult to prevent viral replication in tissues as GCPQ is not absorbed into these areas. The trend towards a reduction of the viral titres in the nasal and respiratory passages provide evidence that GCPQ is likely to limit viral transmission and indeed act as a prophylactic.

### Conclusions

A derivatised chitosan compound with a 6-O-glycol group (lacking in HTCC), a hydrophobic acyl group (lacking in both HTCC and HM-HTCC; the latter derivatised with N-dodecyl groups), a lower molecular weight than HTCC, a trimethyl quaternary ammonium group directly in place of the C2 amine group in chitosan, unlike HTCC (which has the hydroxypropyltrimethylammonium group attached to the C2 nitrogen), and a lower level of quaternary ammonium substitution (<40 mole %) than HTCC, is effectively able to inhibit viral entry into cells. Large structural differences between the prior art and the compounds of the present invention would not lead to the expectation that the compounds of the present invention would exhibit anti-viral activity.

A low molecular weight clearly promotes activity against SARS-COV-2 in mammalian cells (Table 2 and FIGS. 2-3) and this is correlated with the ease with which this polymer may be incorporated into aqueous media. Underivatised glycol chitosan of molecular weights 40-100 kDa were not active (data not shown), demonstrating that quaternary ammonium and possibly palmitoyl groups are important determinants of activity. Furthermore GCPQ possesses advantages for use in viral inhibition and specifically the clinical prevention of viral infections as GCPQ is mucoadhesive, has a long residence time in the nares (FIG. 4) and is chemically stable for at least 18 months. GCPQ also self assembles into nanoparticles and these nanoparticles may be clustered into microparticles for nasal delivery.

GCPQ may therefore be used as a molecular mask nasal spray for the prevention of coronavirus infections. Reduction in brain levels of the virus (FIG. 5) provide encouraging evidence that there is a possibility that the neurological symptoms experienced with SARS-COV-2 infections as reported in The Lancet Neurology (“Long COVID: understanding the neurological effects”, The Lancet Neurol., 20, 247, 2021) may indeed be reduced with the use of the anti-viral prophylactic. After treatment with MMS019, there was a marked inhibition of viral replication in the mouse nasal passages, and decreased levels of the virus were also recorded for the brain tissue, indicating the limited systemic infection. No adverse effects were observed during the experiment.

As GCPQ's activity may not be predicated specifically on the recognition of particular epitopes but appears to be based on electrostatic interactions between GCPQ and the virus, GCPQ may be applied to a wide variety of viral infections. These advantages mean that GCPQ polymer may be used as a nasal spray or by other means for the prevention and treatment of other specific viral infections, in addition to SARS-CoV-2.

1-39. (canceled)

40. A method for the prevention or treatment of a viral infection, comprising administering to a mammal a pharmaceutical composition comprising an amphiphilic carbohydrate compound having the formula: 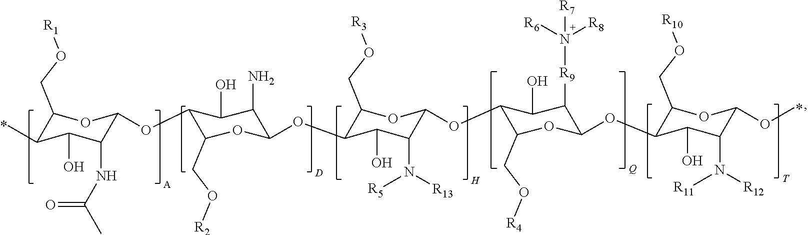
or a salt thereof,
wherein:
the level of unit A is from 0% to 26 mole %;
the level of unit D is from 1% to 95.5 mole %;
the level of unit H is from 1% to 95.5 mole %;
the level of unit Q is from 3% to 40 mole %;
the level of unit T is from 1% to 94.5 mole %;
R₁, R₂, R₃, R₄ and R₁₀ are independently selected from:
hydrogen;
any substituted or unsubstituted alkyl, alkenyl, alkynyl, aryl, or acyl group;
a sugar substituent selected from glucose, galactose, fructose, and muramic acid;
an oligo polyoxa C₁-C₃ alkylene units, optionally substituted with amine, amide or alcohol;
wherein at least one of R₁, R₂, R₃, R₄ and R₁₀ is not hydrogen;
R₅ is a hydrophobic, substituted or unsubstituted, linear, branched or cyclo form of a C₄₋₃₀ alkyl, C₄₋₃₀ alkenyl, C₄₋₃₀ alkynyl, C₄₋₃₀ aryl, C₄₋₃₀ amide, C₄₋₃₀ alcohol or C₃₋₃₀ acyl group;
R₆, R₇, and R₈ are each independently an alkyl group, an alkenyl group, an alkynyl group, an aryl group, or acyl group;
R₉ is absent, a substituted or unsubstituted alkyl group, a substituted or unsubstituted amine group, or a substituted or unsubstituted amide group;
R₁₁ is hydrogen, a substituted or unsubstituted alkyl group, a substituted or unsubstituted ether group, or a substituted or unsubstituted alkene group;
R₁₂ is a substituted or unsubstituted alkyl group, a substituted or unsubstituted ether group, or a substituted or unsubstituted alkene group;
R₁₃ is hydrogen, a substituted or unsubstituted alkyl group, a substituted or unsubstituted ether group, or a substituted or unsubstituted alkene group.

41. The method according to claim 40, wherein the composition is administered intranasally.

42. The method according to claim 40, wherein the viral infection is a coronavirus infection.

43. The method according to claim 40, unit A is in the range 0.5% to 20 mole %.

44. The method according to claim 40, wherein unit D is in the range 2% to 94.5 mole %.

45. The method according to claim 40, wherein unit T is in the range 2% to 94.5 mole %.

46. The method according to claim 40, wherein unit Q is in the range 3% to 30 mole %.

47. The method according to claim 40, wherein unit H is in the range 1% to 20 mole %.

48. The method according to claim 40, wherein each of R₁, R₂, R₃, R₄ and R₁₀ are CH₂CH₂OH.

49. The method according to claim 40, wherein R₅ is C₄₋₃₀ alkyl or a C₃₋₃₀ acyl group.

50. The method according to claim 40, wherein each of R₆, R₇ and R₈ are methyl.

51. The method according to claim 40, wherein R₉ is absent, or R₉ is not a 2-hydroxypropyl group.

52. The method according to claim 40, wherein Ru is H and Ru is methyl, or both of Ru and Ru are methyl.

53. The method according to claim 40, wherein the amphiphilic carbohydrate compound is a quaternary ammonium palmitoyl glycol chitosan (GCPQ).

54. The method according to claim 40, wherein the amphiphilic carbohydrate compound has a molecular weight in the range of 10 to 30 kDa.

55. The method according to claim 40, wherein the amphiphilic carbohydrate compound is in the form of nanoparticles.

56. The method according to claim 40, wherein the composition comprises the amphiphilic carbohydrate compound in a concentration below 50% w/v.

57. The method according to claim 40, wherein the composition further comprises one or more additional active agents.

58. The method according to claim 40, wherein the mammal is human and the composition is administered at a dose of 25 mg/kg to 100 mg/kg.

59. The method according to claim 40, wherein the composition is administered once or twice a day.
